(19) United States
(12) Patent Application Publication
(10) Pub. No.: US 2008/0051448 A1
(43) Pub. Date: Feb. 28, 2008

(54) STABILIZED EXTENDED RELEASE PHARMACEUTICAL COMPOSITIONS COMPRISING AN AMG-COA REDUCTASE INHIBITOR

(75) Inventor: Laxminarayan Joshi, Toronto (CA)

Correspondence Address: PEACOCK MYERS, P.C., 201 THIRD STREET, N.W., SUITE 1340, ALBUQUERQUE, NM, 87102, US

(73) Assignee: Orbus Pharma, Inc., Markhan (CA)

(21) Appl. No.: 11/837,572

(22) Filed: Aug. 13, 2007

Related U.S. Application Data
(63) Continuation-in-part of application No. 11/199,059, filed on Aug. 5, 2005, now abandoned.

(51) Int. Cl. A61K 31/40; A61P 9/00

(52) U.S. Cl. 514/419; 514/789

(57) ABSTRACT

The present invention is a new stable extended release drug composition particularly suitable for use as an antihypercholesterolaemic or antihyperlipidaemic agent. The present invention is specifically a drug composition comprising a pharmaceutical, a complexing agent and a matrix-forming agent, and a method for manufacturing same. When applied to acid-labile drugs like HMG-CoA reductase inhibitors, the resulting drug composition is stabilized and is characterized by an extended-release profile.

### CROSS REFERENCE TO PRIOR APPLICATIONS

This application is a continuation-in-part of U.S. patent application Ser. No. 11/199,059, entitled “Stabilized Extended Release Pharmaceutical Compositions Comprising an HMG-CoA Reductose Inhibitor,” filed on Aug. 5, 2005, and the specification thereof is incorporated herein by reference.

### FIELD OF THE INVENTION

The present invention is a new stable extended release pharmaceutical composition suitable for use as an antihypercholesterolemic or antihyperlipidaemia agent, and more particularly a stable extended release pharmaceutical composition containing as an active substance, an HMG-CoA reductase inhibitor.

### BACKGROUND OF THE INVENTION

Fluvastatin, lovastatin, pravastatin, simvastatin, mevastatin, atorvastatin, and cerivastatin, and derivatives, analogs and pharmaceutically acceptable salts thereof, are known as HMG-CoA reductase inhibitors. They are used as antihypercholesterolemic and antihyperlipidemia agents in humans, and are generally produced by fermentation using microorganisms belonging to any one of the Aspergillus, Monascus, Nocardia, Amycolatopsis, Mucor or Penicillium genus. Some of these inhibitors are obtained by treating the fermentation products using the methods of chemical synthesis (as in the case of simvastatin) or they are the products of total chemical synthesis (as in the cases of fluvastatin, atorvastatin and cerivastatin). Some of these are available as a base (such as lovastatin, simvastatin, mevastatin and cervistatin) while others are available as a salt to improve their aqueous solubility (for example, pravastatin atorvastatin and fluvastatin).

These compounds are particularly sensitive to degradation and destabilization in acidic environments and are typically formulated in a manner that results in rapid delivery. Acidic degradation can be accelerated by interaction with other ingredients like fillers, binders, lubricants, glidants and disintegrating agents. The instability and rapid delivery of an HMG-CoA reductase agent in an acidic environment requires patients to consume higher dosages with greater frequency to achieve the desired therapeutic result, a problem known to result in poor patient compliance.

The degradation kinetics of fluvastatin in aqueous solution at various pH are illustrated in Table 1.

TABLE 1

| | % Fluvastatin sodium | % Fluvastatin sodium |
|---|---|---|
| pH | remains after 1 hr at 37° C. | remains after 24 hr at 37° C. |
| 7.8 | 98.3 | 98.0 |
| 6.0 | 99.6 | 97.1 |
| 4.0 | 86.7 | 25.2 |
| 1.0 | 10.9 | 00.0 |

The instability of HMG-CoA reductase compounds is likely due to the lability of beta, delta-hydroxy groups on a heptanoic acid chain, and the presence of a double bond.

U.S. Pat. No.5,180,589 discloses a degradation resistant formulation for pravastatin by maintaining an alkaline environment with pH above 9, preferably 10. The formulation includes a basifying agent. This solution is problematic because the formulation can have a negative impact on gastric mucosa, especially in patients with damaged gastric mucous membrane.

EP 0,547,000 discloses an alkali carbonate and materials to increase gastric pH above 8.0. However, fluvastatin sodium hygroscopicity results in problematic flow characteristics of the drug and causes problems with encapsulation.

U.S. Pat. No. 6,680,341 discloses HMG-CoA reductase inhibitors protected from pH-related destabilization by the introduction of a buffering agent to the active ingredient. However, the presence of an artificially increased amount of buffering agent in the gastric system can disrupt the body's natural regulatory changes in pH, causing drug absorption problems.

Enteric coatings have also been employed to impede degradation. However, this method requires special care when applying the coating. Enteric coating equipment is expensive, requires high technology workers and is time-intensive.

Extended release drug therapy offers potential advantages, compared with conventional dosage forms such as improving patient compliance, improving clinical efficacy, reducing fluctuations in concentrations of the drug in the blood, and cost effectiveness. There are various methods of manufacturing pharmaceuticals with an extended release profile which provide delivery of a drug over a period of at least six hours. These include methods to control dissolution, diffusion, swelling, osmotic pressure and ion exchange. These methods experience a variety of problems, and range in terms of cost and difficulty in delivery.

Polymeric matrix formulations are one way in which to provide extended release dosage forms containing a therapeutic agent, homogeneously dissolved or dispersed, in a compressed water-swellable core. The mechanism of drug release from polymeric matrices involves solvent penetration, hydration and swelling of the polymer, diffusion of the dissolved drug in the matrix, and erosion of the gel layer. Initially, the diffusion coefficient of the drug in the dehydrated hydrogel is very low, but increases significantly as the gel imbibes water. Whereas interactions between water, polymer, and drug are the primary factors for controlled release, various formulation variables, such as polymer grade, drug/polymer ratio, drug solubility, and drug and polymer particle size can influence drug release rate to a greater or lesser degree.

The selection of the polymeric matrix formation products has been an important first step in extended release formulations, due to the fact that the design of these systems involves the use of polymeric hydration to protect the tablet from rapid disintegration and dissolution in order to delay the release of the drug. Various types of polymers with different solution—gel transitions have been investigated to develop swellable matrices, including hydrophilic cellulose derivaties and polyethylene oxide. The mechanism of extending the release of the drug is governed by the rate-controlling gel layer, which is formed around the solid inner core, in contact with water.

Canadian patent no. 2,346,868 discloses a protective matrix for extended release, manufactured from polyethylene oxide of relatively low molecular weight (meaning 500,000 or less). Such matrix formations have low viscosity and require higher proportions of polyethylene oxide to prepare suitably marketable formulations, resulting in bulkier drug compositions with still relatively poor release profiles, higher manufacturing costs, and poorer overall patient compliance.

Marketable pharmaceutical dosage forms require adequate protection against pH-related destabilization. The composition can be further improved by providing a pharmaceutical with an extended release profile of at least six hours, thus requiring less frequent consumption. The present invention is a stable drug composition having an extended release profile of at least six, and a method for manufacturing same.

### SUMMARY OF THE INVENTION

The present invention provides a stabilized extended-release drug composition comprising a pharmaceutical, a complexing agent and a matrix forming agent.

The present invention further provides a method for manufacturing the above drug composition by providing and mixing together, water and a complexing agent. A pharmaceutical is added to the mixture to form a slurry. The slurry is then dried, and a matrix forming agent is added. Finally, lubricants and fillers are added and the resulting mixture is formed into tablets.

One embodiment of the present invention provides for a drug composition comprising a pharmaceutical, a complexing agent, and a matrix forming agent. For example, a pharmaceutical is a HMG-CoA reductase inhibitor or an acceptable salt thereof. A complexing agent is a cyclodextrin for example. The cyclodextrin can be alpha-cyclodextrin, beta-cyclodextrin, gamma-cyclodextrin or a combination thereof. A matrix forming agent is polyethylene oxide for example. The polyethylene oxide can be ethyl cellulose, or a polyethylene oxide with a molecular weight greater than about 500,000. A pharmaceutical is fluvastatin or an acceptable salt thereof such as fluvastatin sodium for example. A drug composition may be modified with a lubricant, a filler and a combination thereof. A filler can be microcrystalline cellulose and sorbitol.

Another embodiment of the present invention provides for a drug composition comprising a HMG-CoA reductase inhibitor, a cyclodextrin complexing agent; and a matrix forming agent comprising polyethylene oxide having a molecular weight greater than 500,000 and ethyl cellulose.

Yet another embodiment of the present invention provides for a method for manufacture of a drug composition. The method includes mixing water and a complexing agent to form a slurry. To the slurry is added a pharmaceutical, and a filler. The resulting mixture is granulated and the slurry dried. A matrix forming agent is added to the resulting mixture. A lubricant is added to the resulting mixture. The mixture can be formed into tablets. In addition, a hypromellose based coating with titanium dioxide and iron oxide can be added to the tablets.

### BRIEF DESCRIPTION OF THE DRAWINGS

FIG. 1 is an illustration of the structure of beta-cyclodextrin, a complexing agent.

FIG. 2 is an illustration of the complexation of a drug inside a hydrophobic cavity of beta-cyclodextrin.

FIGS. 3A and 3B are illustrations of a stabilized extended release pharmaceutical composition in a non-eroding matrix formulation in relaxed and swollen forms, respectively.

### DETAILED DESCRIPTION

Pharmaceutical compositions containing HMG-CoA reductase inhibitors (such as statins and acceptable statin salts) are stable at basic pH levels. Higher pH levels, preferably greater than 9, yield more stable pharmaceutical grade HMG-CoA reductase inhibitors. Acidic environment like gastric mucosa rapidly destabilize and disintegrate HMG-CoA reductase inhibitors Rapid destabilization and disintegration requires patients to consume higher dosages with greater frequency, resulting in poor patient compliance and greater frequency of adverse and side effects.

In a preferred embodiment of the present invention, a pharmaceutical HMG-CoA reductase inhibitor (for example, fluvastatin sodium) is protected against destabilization in an acidic environment by utilizing cyclodextrin, and more preferably beta-cyclodextrin, as an inclusion complexing agent. This drug composition is then subjected to a matrix forming agent resulting in an extended release profile of at least six hours.

Complexation, the reversible association of a substrate and ligand to form a new species, is one way to favorably enhance the physicochemical properties of pharmaceutical compounds. Cyclodextrins are examples of compounds that form inclusion complexes. These complexes are formed when a “guest” molecule is partially or fully included inside a “host” molecule with no covalent bonding. When inclusion complexes are formed, the physicochemical parameters of the guest molecule are disguised or altered, and improvements in the molecule's solubility, stability, taste, safety and bioavailability are commonly seen.

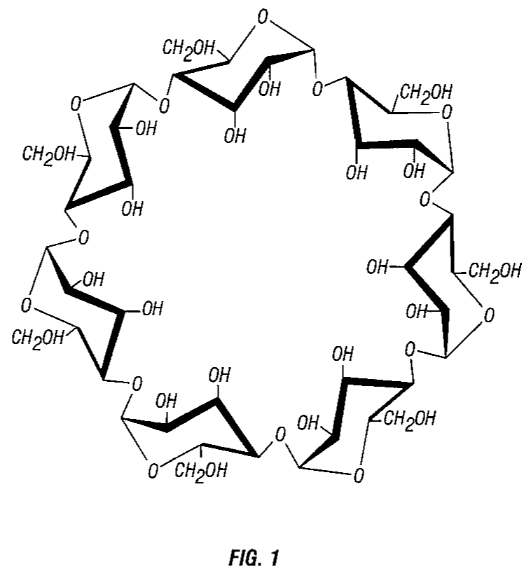

Cyclodextrins are cyclic oligosaccharides containing 6, 7, or 8 glucopyranose units, referred to as alpha, beta or gamma cyclodextrin, respectively. Each glucose unit contains two secondary alcohols at C-2 and C-3, and a primary alcohol at the C-6 position, providing 18-24 sites for chemical modification and derivatization. The chemical structure of beta-cyclodextrin is shown in FIG. 1.

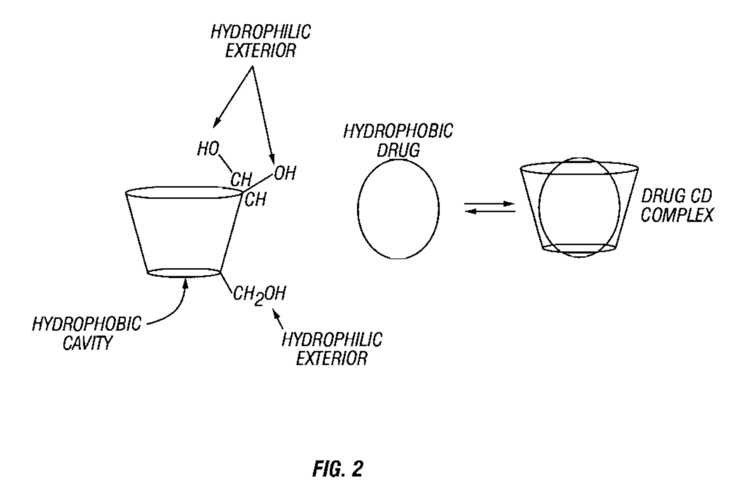

FIG. 2 shows cyclodextrin defining a hydrophobic cavity relative to an aqueous environment. Sequestration of hydrophobic drugs inside the cyclodextrin cavity can improve a drug's solubility and stability in water, the rate and extent of dissolution of the drug:cyclodextrin complex, and the bioavailability of the drug when dissolution and solubility are limiting the delivery. These cyclodextrin properties enable insoluble drug formulations that are typically difficult to formulate and deliver with more traditional excipients.

A cyclodextrin inclusion complex is resistant to hydrolysis in the acidic environment of the stomach, thus maintaining an active drug ingredient as a guest within the inclusion complex following oral administration. This permits the active drug ingredient to pass through the stomach and resist degradation and destabilization in the acidic environment of the stomach. However, the inclusion complex is not resistant to digestion by enzymes present in the intestinal region, thus causing its breakdown and the release of the active drug ingredient for absorption. In some cases, the drug is released from the inclusion complex upon dilution with contributions from competitive displacement with endogenous lipophiles binding to plasma and tissue components where drug uptake into tissues is not available to the complex and the beta-cyclodextrin is rapidly eliminated.

Applying a matrix to the inclusion complex results in extended release of the active drug into its target. The application of a non-eroding matrix using polyethylene oxide having a relatively high molecular weight results in an extended release of drug over a period of at least six hours.

In a preferred embodiment of the present invention, the matrix includes polyethylene oxide having a molecular weight greater than 500,000, preferably greater than 5,000,000, and preferably about 7,000,000. Ethyl cellulose is also selected as a matrix forming agent. The matrix can be further improved by granulation with a filler, such as microcystalline cellulose or sorbitol. The high viscosity of these ingredients (in particular the high molecular weight polyethylene oxide) results in the formation of a strong non-eroding matrix, which is preferred for use with highly soluble drugs, such as Fluvastatin.

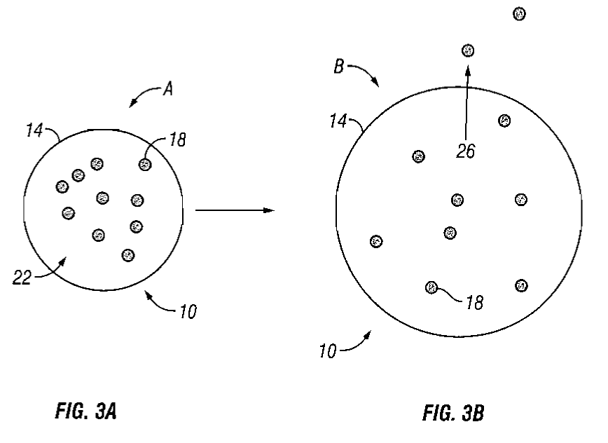

FIGS. 3A and 3B show a stabilized extended release pharmaceutical composition (10) in a non-eroding matrix formulation (14) in relaxed and swollen forms, respectively. When a dosage form containing a drug (18) (e.g. complexed HMG-CoA reductase inhibitor) in a matrix formulation (14) is ingested and exposed to a gastric environment (FIG. 3A), dissolution material, such as gastric fluids (22), enters into the tablet matrix (14) causing the form to swell to capacity (FIG. 3B), preventing rapid release of the drug (18). During the initial period following exposure, leeching (26) of complexed drug (18) from the swollen tablet matrix (FIG. 3B) occurs. This allows for the commencement of the therapeutic effects of the drug (18) without delay. The complexation of the drug (18) stabilizes the leeched drug (26) while in the gastric environment, allowing the drug to pass through the intestines (not shown) where it is released from complexation and absorbed. This release mechanism continues over an extended period providing the desired extended release profile.

Manufacture of a preferred embodiment of the present invention is achieved using the following steps (which are provided for example purposes only):

| Number | Step |
|---|---|
| 1. | transfer a calculated amount of water to a stainless steel vessel |
| | fitted with a mechanical stirrer; |
| 2. | slowly stir in a desired amount of complexing agent, such as |
| | beta cyclodextrin, in small lots; |
| 3. | add the desired amount of HMG-CoA reductase inhibitor, |
| | such as fluvastatin sodium, in small lots; |
| 4. | granulate the resulting mixture with microcrystalline cellulose |
| | as a filler; |
| 5. | dry the granulated mass and screened through a mesh; |
| 6. | mix in the desired amount of polyethylene oxide, Magnesium |
| | stearate and ethyl cellulose 100 cps; |
| 7. | compress the resulting mixture into tablets; and |
| 8. | coat the tablets with Hypromellose based coating including |
| | titanium oxide and iron oxide. |

In furtherance of the example above, the following 80 mg dosages of Fluvastatin can be manufactured using the following amounts of the listed ingredients:

### EXAMPLE 1

| S. NO. | INGREDIENTS | QUANTITY/TABLET | |
|---|---|---|---|
| 1 | FLUVASTATIN SODIUM | 84.48 MG = 80.00 MG | |
| 2 | BETADEX | 126.72 | MG |
| 3 | MICROCRYSTALLINE | 130.00 | MG |
| | CELLULOSE | | |
| 4 | POLYETHYLENE OXIDE | 50.00 | MG |
| 5 | ETHYL CELLULOSE 100 CPS | 20.00 | MG |
| 6 | MAGNESIUM STEARATE | 10.00 | MG |
| 7 | SPECTRAFILM YELLOW | 17.00 | MG |

| | | |
|---|---|---|
| 8 | WATER | Q.S |

| | |
|---|---|
| 438.2 | MG/TABLET |

### EXAMPLE 2

| S. NO. | INGREDIENTS | QUANTITY/TABLET | |
|---|---|---|---|
| 1 | FLUVASTATIN SODIUM | 84.48 MG = 80.00 MG | |
| 2 | BETADEX | 126.72 | MG |
| 3 | MICROCRYSTALLINE | 130.00 | MG |
| | CELLULOSE | | |
| 4 | POLYETHYLENE OXIDE | 55.00 | MG |
| 5 | ETHYL CELLULOSE 100 CPS | 20.00 | MG |
| 6 | MAGNESIUM STEARATE | 10.00 | MG |
| 7 | SPECTRAFILM YELLOW | 17.00 | MG |

| | | |
|---|---|---|
| 8 | WATER | Q.S |

| | |
|---|---|
| 443.2 | MG/TABLET |

### EXAMPLE 3

| S. NO. | INGREDIENTS | QUANTITY/TABLET | |
|---|---|---|---|
| 1 | FLUVASTATIN SODIUM | 84.48 MG = 80.00 MG | |
| 2 | BETADEX | 126.72 | MG |
| 3 | MICROCRYSTALLINE | 130.00 | MG |
| | CELLULOSE | | |
| 4 | POLYETHYLENE OXIDE | 55.00 | MG |
| 5 | ETHYL CELLULOSE 100 CPS | 25.00 | MG |
| 6 | MAGNESIUM STEARATE | 10.00 | MG |
| 7 | SPECTRAFILM YELLOW | 17.00 | MG |

| | | |
|---|---|---|
| 8 | WATER | Q.S |

| | |
|---|---|
| 448.2 | MG/TABLET |

Example 3 results in a release profile of at least six hours, as illustrated by the following release profile table:

| Time after ingestion | Amount of Drug Released |
|---|---|
| (hours) | (as % of initial amount of drug) |
| 1.0 | 6% |
| 3.0 | 26% |
| 6.0 | 65% |
| 12.0 | 100% |

While the subject invention has been described and illustrated with reference to certain particular embodiments thereof, those skilled in the art will appreciate that various adaptations, changes, modifications, substitutions, deletions or additions of procedures and protocols may be made without departing from the scope of the invention.

What is claimed is:

1. A drug composition comprising:
fluvastatin;
a complexing agent; and
a matrix forming agent.

2. The drug composition as claimed in claim 1 wherein the fluvastatin is fluvastatin sodium.

3. The drug composition as claimed in claim 1 wherein the complexing agent is a cyclodextrin.

4. The drug composition as claimed in claim 3 wherein the cyclodextrin is chosen from one of a group of alpha-cyclodextrin, beta-cyclodextrin and gamma-cyclodextrin.

5. The drug composition as claimed in claim 1 wherein the matrix forming agent is polyethylene oxide.

6. The drug composition as claimed in claim 5 wherein the matrix forming agent further includes ethyl cellulose.

7. The drug composition as claimed in claim 7 wherein the polyethylene oxide has a molecular weight greater than 500,000.

8. The drug composition as claimed in claim 1 further comprising a lubricant and a filler.

9. The drug composition as claimed in claim 1 further comprising a lubricant and a filler, wherein the fluvastatin is fluvastatin sodium, the complexing agent is beta-cyclodextrin, a matrix forming agent is polyethylene oxide with a molecular weight greater than 500,000 and wherein a matrix forming agent further includes ethyl cellulose.

10. A drug composition comprising:
fluvastatin;
a cyclodextrin complexing agent; and
matrix forming agents comprising polyethylene oxide having a molecular weight greater than 500,000 and ethyl cellulose.

11. A method for manufacture of a drug composition comprising:
mixing water and a complexing agent to form a slurry;
adding a pharmaceutical to the slurry;
adding a filler to the slurry;
granulating the resulting mixture;
drying the slurry;
adding a matrix forming agent to the resulting mixture: and
adding a lubricant to the resulting mixture.

12. The method as claimed in claim 11 wherein the pharmaceutical is fluvastatin sodium.

13. The method as claimed in claim 12 wherein the complexing agent is cyclodextrin.

14. The method as claimed in claim 13 wherein the filler is chosen from the group consisting of microcrystalline cellulose and sorbitol.

15. The method as claimed in claim 14 wherein the matrix forming agent is polyethylene oxide.

16. The method as claimed in claim 15 wherein the polyethylene oxide has a molecular weight greater than 500,000.

17. The method as claimed in claim 16 wherein the matrix forming agent further includes ethyl cellulose.

18. The method as claimed in claim 11 further comprising:
forming the resulting mixture into tablets.

19. The method as claimed in claim 18 further comprising:
applying a hyprommellose based coating with titanium dioxide and iron oxide to the tablets.
